(19) United States
(12) Patent Application Publication
(10) Pub. No.: US 2004/0241209 A1
(43) Pub. Date: Dec. 2, 2004

(54) Carbonated candy-type pharmaceutical preparation

(76) Inventor: Won-Gyu Kim, Seoul (KR)

Correspondence Address: BIRCH STEWART KOLASCH & BIRCH, PO BOX 747, FALLS CHURCH, VA, 22040-0747, US

(21) Appl. No.: 10/618,818

(22) Filed: Jul. 15, 2003

(30) Foreign Application Priority Data
May 30, 2003 (KR) 10-2003-0034614

(51) Int. Cl. A61K 9/68

(52) U.S. Cl. 424/440

(57) ABSTRACT

The present invention is concerned with a new carbonated candy-type vitamin preparation comprising at least one active ingredient selected from the group consisting fat-soluble vitamins, water soluble vitamins, amino acids, trace inorganic compound, other ingredients necessary for human beings, organic acids; sucrose and; starch syrup(maltose syrup); sodium bicarbonate; and carbon dioxide, with the proviso that at least one vitamin is comprised. Children eagerly take the present vitamin preparation and when the preparation is taken in the mouth, the carbonated pharmaceutical vitamin preparation is popped out and the active ingredients are diffused and dissolved in the mouth very quickly and therefore, the effects of the active ingredients showed very quickly.

### TITLE OF THE INVENTION

A new carbonated candy-type pharmaceutical preparation

### BRIEF DESCRIPTION OF DRAWINGS

FIG. 1 is a flow chart showing the manufacturing process for carbonated candy-type pharmaceutical preparation of the present invention,

FIG. 2 is an another flow chart showing the manufacturing process for a large carbonated candy-type pharmaceutical preparation of the present invention.

### DETAILED DESCRIPTION OF THE INVENTION

[Purpose of the Invention]

[Background of the Invention]

The present invention concerned with a new type of pharmaceutical preparation. Especially, the present invention concerned with a new carbonated candy-type vitamin preparation for children.

A multi-vitamins preparation comprises various vitamins, amino acids, trace inorganic compounds, collagen, green tea extract, ginseng radix extract and other ingredients necessary for human beings. Such preparation is manufactured and sold in the form of a known pharmaceutical preparation such as tablet, capsule, film-coated tablet or soft capsule etc. Though vitamins are necessary for children and weak people, such a preparation as the said form is however difficult for children to take it. Carbonated candy is a hard candy form composed mainly with sugar and containing carbon dioxide as disclosed in U.S. Pat. No. 3,012,893 which is herein incorporated by reference. The carbonated candy when placed in the mouth produces an entertaining but short lived popping sensation and is popular eating between meals for children.

[Technical Subject to be Accomplished by the Invention]

The invention concerned with a new carbonated candy-type vitamin preparation for children and weak people. The inventor has studied for a long time to solve problems of the known vitamin preparation and finally found out the fact that, when vitamin preparation is prepared in the form of carbonated candy-type incorporated with carbon dioxide, children eagerly take the preparation and when the preparation is taken in the mouth, the preparation is popped out and the active ingredients are diffused and dissolved in the mouth very quickly and therefore, the effects of the active ingredients showed very quickly.

[Constitution of the Invention]

The present invention concerned with a carbonated candy-type vitamin preparation comprising at least one vitamin selected from the group consisting vitamins, trace inorganic compounds, ingredients necessary for human beings, organic acids, amino acids; sucrose; starch syrup(maltose syrup); lactose; carbon dioxide; sodium hydrogen carbonate; and prepared by a conventional manufacturing process for carbonated candy.

As for vitamins which can be used in the present invention, fat-soluble vitamins such as vitamin A₁(retinol), vitamin A₂(3-dehydroretinol), vitamin A₃, vitamin D₂(ergocalciferol), vitamin D₃(cholecalfeferol), provitamin D₂(ergosterin), provitamin D₃(dehydrocholesterol), vitamin E(α-tocopherol, β-tocopherol, γ-tocopherol, δ-tocopherol), vitamin F(linoleic acid, linolenic acid), vitamin K₁, vitamin K₂ or vitamin U, water-soluble vitamins such as vitamin B₁(thiamine), vitamine B₂(riboflavin), vitamin B₆(pyridoxin), nicotinamide, nicotinic acid, pantothenic acid, vitamin H(biotin), folic acid, vitamin B₁₂(cyanocobalamin), choline, inositol, vitamin L₁(anthranilic acid), vitamin L₂(5′-thiomethyladenosine), vitamin B₁₃(orotic acid) or vitamin C(ascorbic acid) can be exemplified. As for amino acid, glycine, alanine, valine, leucine, isoleucine, serine, threonine, cysteine, methionine, asparaginic acid, glutaminic acid, lysine, arginine, phenylalanine, tyrosine, histidine, tryptophane, proline and oxyproline can be exemplified. Other ingredients such as KI, MgO, Cu₂O, ZnSO₄, MnSO₄, CaHPO₄, KCl, dried yeast(yeast containing Cr, yeast containing Se, yeast containing Mo), precipitated calcium carbonate, pomegranate extract, collagen, chitosan, green tea extract, Ginseng Radix extract, Acanthopanacis Cortex extract, onion extract, malic acid, citric acid, tartaric acid, fumaric acid, maleic acid or acetic acid can be used. Other ingredient such as fruit essence, fruit extract, food color or emulsifier can be added.

In the present invention, 1-30 weight parts of active ingredient, 3-30 weight parts of sucrose, 3-30 weight parts of starch syrup(maltose syrup), suitable amount of ingredient selected from the group consisting emulsifier, fruit juice, fruit essence or food color can be mixed and processed by a conventional carbonated candy manufacturing method to obtain a carbonated candy-type vitamin preparation.

The preparation method can be explained as follows:

1. Active ingredients, sucrose, starch syrup(maltose syrup) and other ingredients, except carbon dioxide, sodium bicarbonate and color are dissolved, suspended or emulsified in a suitable amount of water and the mixture is heated rapidly to obtain a molten mass;

2. The molten mass is concentrated to about 1-3 weight parts of water to obtain a concentrated molten mass;

3. The concentrated molten mass is transferred to a pre-heated autoclave where color, essence and sodium bicarbonate are added by rapid stirring and is injected with carbon dioxide gas by rapid stirring to disperse the carbon dioxide bubbles under high pressure;

4. The molten carbon-dioxide-gasified mass is injected into a suitable size of tube or candy-type mold;

5. The tube or mold injected with molten carbon-dioxide-gasified mass is cooled to or below 15° C.;

6. The cooled carbonated candy-type mass is taken out and/or crushed under or below 20° C., under or below 40% of RH;

7. In the case of crushed carbonated mass, the crushed carbonated mass is sieved through a standard sieve; and

8. The carbonated candy-type mass or sieved crushed carbonated mass is sealed and packaged in a hermetic package.

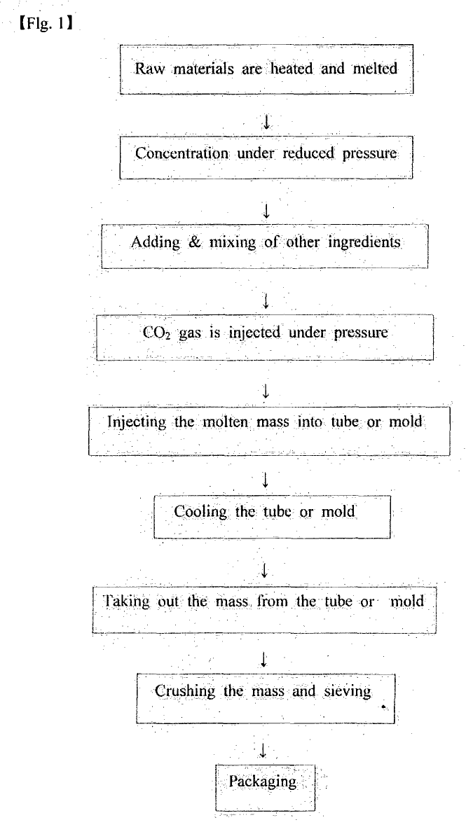

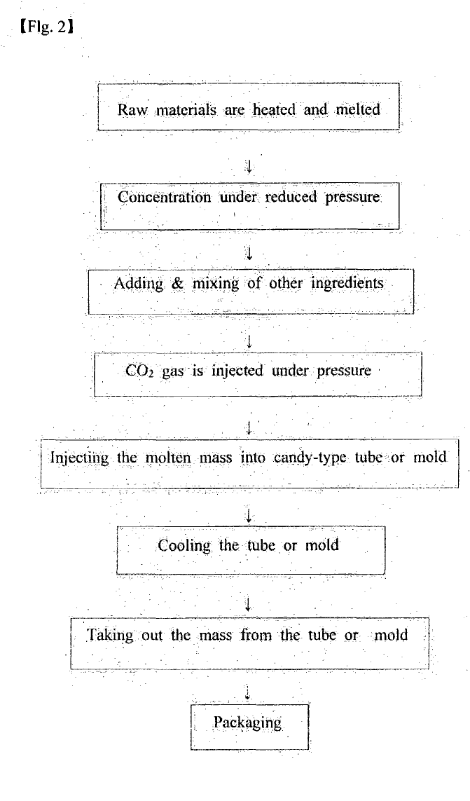

The above procedure is showed in FIG. 1. FIG. 2 is an another flow chart showing the manufacturing process for a large carbonated candy-type pharmaceutical preparation of the present invention.

The present invention is explained in more detail by the examples.

### EXAMPLE 1

| | | |
|---|---|---|
| vitamin C | 250 | g |
| collagen powder | 200 | g |
| pomegranate extract powder | 20 | g |
| lysine | 200 | g |
| sucrose | 700 | g |
| starch syrup (maltose syrup) | 400 | g |
| lactose | 400 | g |
| carbon dioxide | 34 | g |
| sodium bicarbonate | 20 | g |
| grape essence | 50 | g |
| food color, Blue No. 1 | 6.4 | g |
| food color, Red No. 1 | 2 | g |

In a tank 400 ml of water, vitamin C, collagen powder, pomegranate extract powder, lysine, sucrose, starch syrup(maltose syrup) and lactose are added under stirring and the mixture is heated to have molten mass. The molten mass is deaerated and concentrated to a mass having about 2 weight % of water content at the same temperature. To the concentrated molten mass, remaining ingredients are added and carbon dioxide is injected at carbon dioxide pressure of 40^{˜}50 kg/cm² by rapid stirring to disperse the gas bubbles. After stirring, the gasified molten mass is injected into a suitable metal tube. The tube or mold injected with molten mass is cooled to or below 15° C. The cooled carbonated candy-type mass is taken out and crushed under or below 20° C., under or below 40% of RH. The crushed carbonated mass is sieved through a standard sieve. About 2.0 g of the carbonated candy-type mass or sieved crushed carbonated mass sealed and packaged in a hermetic aluminium film package coated with polypropylene film.

### EXAMPLE 2

| | | |
|---|---|---|
| vitamin C | 500 | g |
| vitamin B₁ | 200 | g |
| collagen powder | 200 | g |
| pomegranate extract powder | 20 | g |
| sucrose | 500 | g |
| starch syrup (maltose syrup) | 400 | g |
| lactose | 400 | g |
| carbon dioxide | 34 | g |
| sodium bicarbonate | 20 | g |
| grape essence | 50 | g |
| food color, Blue No. 1 | 6.4 | g |
| food color, Red No. 1 | 2 | g |

400 ml of water, vitamin C, vitamin B₁, collagen powder, pomegranate extract powder, sucrose, maltose syrup and lactose are added under stirring in a tank and the mixture is heated to have molten mass. The molten mass is deaerated and concentrated to a mass having about 2 weight % of water content at the same temperature. To the concentrated molten mass, remaining ingredients are added and carbon dioxide is injected at carbon dioxide pressure of 40^{˜}50 kg/cm² by rapid stirring to disperse the gas bubbles. After stirring, the gassified molten mass is injected into a suitable metal tube. The tube or mold injected with molten mass is cooled to or below 15° C. The cooled carbonated candy-type mass is taken out and crushed under or below 20° C., under or below 40% of RH. The crushed carbonated mass is sieved through a standard sieve. About 2.0 g of the carbonated candy-type mass or sieved crushed carbonated mass sealed and packaged in a hermetic aluminium film package coated with polypropylene film.

### EXAMPLE 3

| | | |
|---|---|---|
| vitamin C | 1000 | g |
| vitamin B₁ | 200 | g |
| collagen powder | 200 | g |
| pomegranate extract powder | 20 | g |
| sucrose | 500 | g |
| starch syrup (maltose syrup) | 400 | g |
| lactose | 400 | g |
| carbon dioxide | 34 | g |
| sodium bicarbonate | 20 | g |
| strawberry essence | 50 | g |
| food color, Blue No. 1 | 6.4 | g |
| food color, Red No. 1 | 2 | g |

A carbonated candy-type vitamin preparation is prepared by a similar method to Example 1.

### EXAMPLE 4

| | | |
|---|---|---|
| vitamin C | 1000 | g |
| riboflavin | 10 | g |
| pyridoxine.HCl | 25 | g |
| vitamin B₁ | 200 | g |
| collagen powder | 200 | g |
| green tea extract powder | 200 | g |
| Acanthopanacis Cortex extract powder | 100 | g |
| alanine | 50 | g |
| valine | 50 | g |
| sucrose | 500 | g |
| starch syrup (maltose syrup) | 400 | g |
| lactose | 400 | g |
| carbon dioxide | 34 | g |
| sodium bicarbonate | 20 | g |
| orange essence | 50 | g |
| food color, Blue No. 1 | 6.4 | g |
| food color, Red No. 1 | 2 | g |

A carbonated candy-type vitamin preparation is prepared by a similar method to Example 1.

### EXAMPLE 5

| | | |
|---|---|---|
| vitamin C | 250 | g |
| riboflavin | 10 | g |
| pyridoxine.HCl | 25 | g |
| vitamin B₁ | 50 | g |
| tocopherol acetate | 50 | g |
| dried yeast containing Se | 50 | g |
| Ginseng Radix extract | 100 | g |
| sucrose | 750 | g |
| starch syrup (maltose syrup) | 400 | g |
| lactose | 400 | g |
| carbon dioxide | 34 | g |
| sodium hydrogen carbonate | 20 | g |
| orange essence | 50 | g |
| food color; Blue No. 1 | 6.4 | g |
| food color, Red No. 1 | 2 | g |

A carbonated, candy-type vitamin preparation is prepared by a similar method to Example 1.

### EXAMPLE 6

| | | |
|---|---|---|
| vitamin C | 250 | g |
| riboflavin | 10 | g |
| pyridoxine.HCl | 25 | g |
| vitamin B₁ | 50 | g |
| tocopherol acetate | 50 | g |
| dried yeast containing Se | 50 | g |
| retinol acetate | 20 | g |
| ergocalciferol | 10 | g |
| Acanthopanacis Cortex extract powder | 100 | g |
| sucrose | 750 | g |
| starch syrup (maltose syrup) | 400 | g |
| lactose | 400 | g |
| carbon dioxide | 34 | g |
| sodium bicarbonate | 20 | g |
| orange essence | 50 | g |
| food color, Yellow No. 4 | 5 | g |
| food color, Red No. 3 | 2.5 | g |

A carbonated candy-type vitamin preparation is prepared by a similar method to Example 1.

### EXAMPLE 7

| | | |
|---|---|---|
| vitamin C | 500 | g |
| riboflavin | 50 | g |
| pyridoxine.HCl | 55 | g |
| vitamin B₁ | 250 | g |
| tocopherol acetate | 50 | g |
| isoleucine | 10 | g |
| serine | 10 | g |
| threonine | 10 | g |
| dried yeast containing Se | 50 | g |
| retinol acetate | 20 | g |
| ergocalciferol | 10 | g |
| Acanthopanacis Cortex extract | 100 | g |
| KI | 10 | g |
| sucrose | 750 | g |
| starch syrup (maltose syrup) | 400 | g |
| lactose | 400 | g |
| carbon dioxide | 34 | g |
| sodium bicarbonate | 20 | g |
| orange essence | 50 | g |
| food color, Yellow No. 4 | 5 | g |
| food color, Red No. 3 | 2.5 | g |

A carbonated candy-type vitamin preparation is prepared by a similar method to Example 1.

### EXAMPLE 8

| | | |
|---|---|---|
| vitamin C | 500 | g |
| riboflavin | 50 | g |
| pyridoxine.HCl | 5 | g |
| vitamin B₁ | 250 | g |
| tocopherol acetate | 50 | g |
| dried yeast containing Se | 50 | g |
| retinol acetate | 20 | g |
| ergocalciferol | 10 | g |
| Acanthopanacis Cortex extract | 100 | g |
| KI | 10 | g |
| sucrose | 750 | g |
| starch syrup (maltose syrup) | 400 | g |
| lactose | 400 | g |
| carbon dioxide | 34 | g |
| sodium bicarbonate | 20 | g |
| orange essence | 50 | g |
| food color, Yellow No. 4 | 5 | g |
| food color, Red No. 3 | 2.5 | g |

400 ml of water, vitamin C, vitamin B₁, riboflavin, pyridoxin.HCl, tocopherol acetate, dried yeast containing Se, retinol acetate, ergocalciferol, Acanthopanacis Cortex extract, KI, sucrose, starch syrup(maltose syrup) and lactose are added in a tank under stirring and the mixture is heated to have molten mass. The molten mass is deaerated and concentrated to a mass having about 2 weight % of water content at the same temperature. To the concentrated molten mass, remaining ingredients are added and carbon dioxide gas is injected at carbon dioxide pressure of 40^{˜}50 kg/cm² by rapid stirring to disperse the gas bubbles. After stirring, the gasified molten mass is injected into a candy-type mold of 2 g content. The mold injected with molten mass is cooled to or below 15° C. The cooled carbonated candy-type mass is taken out under or below 20° C., under or below 40% of RH. The carbonated candy-type mass sealed and packaged in a hermetic aluminium film package coated with polypropylene film.

### EXAMPLE 9

| | | |
|---|---|---|
| vitamin C | 250 | g |
| riboflavin | 50 | g |
| pyridoxine.HCl | 55 | g |
| vitamin B₁ | 250 | g |
| tocopherol acetate | 50 | g |
| dried yeast containing Se | 50 | g |
| retinol acetate | 20 | g |
| ergocalciferol | 10 | g |
| KI | 10 | g |
| arginine | 10 | g |
| histidine | 10 | g |
| onion extract | 50 | g |
| chitosan | 100 | g |
| citric acid | 50 | g |
| sucrose | 750 | g |
| starch syrup (maltose syrup) | 400 | g |
| lactose | 400 | g |
| carbon dioxide | 34 | g |
| sodium bicarbonate | 20 | g |
| orange essence | 50 | g |
| food color, Yellow No. 4 | 5 | g |
| food color, Red No. 3 | 2.5 | g |

A carbonated candy-type vitamin preparation is prepared by a similar method to Example 8.

### EXAMPLE 10

| | | |
|---|---|---|
| vitamin C | 250 | g |
| riboflavin | 50 | g |
| pyridoxine.HCl | 55 | g |
| vitamin B₁ | 250 | g |
| glycine | 10 | g |
| leucine | 20 | g |
| tocopherol acetate | 50 | g |
| dried yeast containing Se | 50 | g |
| retinol acetate | 20 | g |
| ergocalciferol | 10 | g |
| KI | 10 | g |
| onion extract | 50 | g |
| emulsifier lecithin | 45 | g |
| citric acid | 50 | g |
| sugar | 750 | g |
| starch syrup (maltose syrup) | 400 | g |
| lactose | 400 | g |
| carbon dioxide | 34 | g |
| sodium bicarbonate | 20 | g |
| orange essence | 50 | g |
| food color, Yellow No. 4 | 5 | g |
| food color, Red No. 3 | 2.5 | g |

A carbonated candy-type vitamin preparation is prepared by a similar method to Example 8.

[Effect of the Invention]

The present invention concerned with a carbonated candy-type vitamin preparation comprising at least one vitamin selected from the group consisting vitamins, sucrose, starch syrup(maltose syrup), lactose, carbon dioxide, sodium bicarbonate and prepared by a conventional manufacturing process for carbonated candy. The present preparation can have, if necessary, one or more ingredients selected from the group consisting of trace elements or other ingredients necessary for children or weak people. Children eagerly take the preparation and when the preparation is taken in the mouth, the carbonated pharmaceutical vitamin preparation is popped out and the active ingredients are diffused and dissolved in the mouth very quickly and therefore, the effects of the active ingredients showed very quickly.

What is claimed is:

1. A new carbonated candy-type vitamin preparation comprising at least one active ingredient selected from the group consisting fat-soluble vitamins and water soluble vitamins, trace inorganic compounds, amino acids, organic acids, and other ingredients necessary for human being; sucrose and; starch syrup(maltose syrup), sodium bicarbonate; and carbon dioxide with the proviso that at least one vitamin is comprised, characterized in that the carbonated candy-type vitamin preparation is manufactured by
1) Active ingredients, sucrose, starch syrup(maltose syrup) and other ingredients, except carbon dioxide and sodium bicarbonate are dissolved, suspended or emulsified in suitable amount of water and the mixture is heated rapidly to obtain a molten mass;
2) The molten mass is concentrated to about 1-3 weight parts of water;
3. The concentrated molten mass is transferred to a pre-heated autoclave where color, essence and sodium bicarbonate are added by rapid stirring and is injected with carbon dioxide gas by rapid stirring to disperse the carbon dioxide bubbles under high pressure;
4) The molten carbon-dioxide-gasified mass is injected into a suitable size of tube or candy-type mold;
5) The tube or mold injected with molten carbon-dioxide-gasified mass is cooled to or below 15° C.;
6) The cooled carbonated candy-type mass is taken out and/or crushed under or below 20° C., under or below 40% of RH;
7) In the case of crushed carbonated mass, the crushed carbonated mass is sieved through a standard sieve; and
8) The carbonated candy-type mass or sieved crushed carbonated mass is sealed and packaged in a hermetic package.

2. A new carbonated candy-type vitamin preparation of claim 1 wherein least one active ingredient is selected from the group consisting fat-soluble vitamins and water soluble vitamins, trace element, trace ingredients is selected from the group consisting vitamin A₁(retinol), vitamin A₂(3-dehydroretinol), vitamin A₃, vitamin D₂(ergocalciferol), vitamin D₃(cholecalfeferol), provitamin D₂(ergosterin), provitamin D₃(dehydrocholesterol), vitamin E(α-tocopherol, β-tocopherol, γ-tocopherol, δ-tocopherol), vitamin F(linoleic acid, linolenic acid), vitamin K₁, vitamin K₂, vitamin U, vitamin B₁(thiamine), vitamine B₂(riboflavin), vitamin B₆(pyridoxin), nicotinamide, nicotinic acid, pantothenic acid, vitamin H(biotin), folic acid, vitamin B₁₂(cyanocobalamin), choline, inositol, vitamin L₁(anthranilic acid), vitamin L₂(5′-thiomethyladenosine), vitamin B₁₃(orotic acid), vitamin C(ascorbic acid), glycine, alanine, valine, leucine, isoleucine, serine, threonine, cysteine, methionine, asparaginic acid, glutaminic acid, lysine, arginine, phenylalanine, tyrosine, histidine, tyiptophane, proline, oxyproline, lactose, fructose, glucose, KI, MgO, Cu₂O, ZnSO₄, MnSO₄, CaHPO₄, KCl, dried yeast(yeast containing Cr, yeast containing Se, yeast containing Mo), precipitated calcium carbonate, pomegranate extract, collagen, chitosan, green tea extract, Ginseng Radix extract, Acanthopanacis Cortex extract, onion extract, malic acid, citric acid, tartaric acid, fumaric acid, maleic acid and acetic acid.

3. A process for the preparation of a new carbonated candy-type vitamin preparation comprising at least one active ingredient selected from the group consisting fat-soluble vitamins and water soluble vitamins, tracee inorganic compound, trace ingredients; sucrose and; starch syrup(maltose syrup), sodium bicarbonate; and carbon dioxide, with the proviso that at least one vitamin is comprised, characterized in that the carbonated candy-type vitamin preparation is manufactured by
1) Active ingredients, sucrose, starch syrup(maltose syrup) and other ingredients, except carbon dioxide and sodium bicarbonate are dissolved, suspended or emulsified in suitable amount of water and the mixture is heated rapidly to obtain a molten mass;
2) The molten mass is concentrated to about 1-3 weight parts of water;
3) The concentrated molten mass is transferred to a pre-heated autoclave where the molten mass is added sodium hydrogen carbonate by rapid stirring and is injected with carbon dioxide gas by rapid stirring to disperse the carbon dioxide gas bubbles under high pressure;
4) The molten carbon-dioxide-gasified mass is injected into a suitable size of tube or candy-type mold;
5) The tube or mold injected with molten carbon-dioxide-gasified mass is cooled to or below 15° C.;
6) The cooled carbonated candy-type mass is taken out and/or crushed under or below 20° C., under or below 40% of RH;
7) In the case of crushed carbonated mass, the crushed carbonated mass is sieved through a standard sieve; and
8) The carbonated candy-type mass or sieved crushed carbonated mass is sealed and packaged in a hermetic package.

4. A process for the preparation of a new carbonated candy-type vitamin preparation of claim 1 wherein least one active ingredient is selected from the group consisting fat-soluble vitamins and water soluble vitamins, trace element, trace ingredients is selected from the group consisting vitamin A₁(retinol), vitamin A₂(3-dehydroretinol), vitamin A₃, vitamin D₂(ergocalciferol), vitamin D₃(cholecalfeferol), provitamin D₂(ergosterin), provitamin D₃(dehydrocholesterol), vitamin E(α-tocopherol, β-tocopherol, γ-tocopherol, δ-tocopherol), vitamin F(linoleic acid, linolenic acid), vitamin K₁, vitamin K₂, vitamin U, vitamin B₁(thiamine), vitamine B₂(riboflavin), vitamin B₆(pyridoxin), nicotinamide, nicotinic acid, pantothenic acid, vitamin H(biotin), folic acid, vitamin B₁₂(cyanocobalamin), choline, inositol, vitamin L₁(anthranilic acid), vitamin L₂(5′-thiomethyladenosine), vitamin B₁₃(orotic acid), vitamin C(ascorbic acid), glycine, alanine, valine, leucine, isoleucine, serine, threonine, cysteine, methionine, asparaginic acid, glutaminic acid, lysine, arginine, phenylalanine, tyrosine, histidine, tyiptophane, proline, oxyproline, lactose, fructose, glucose, KI, MgO, Cu₂O, ZnSO₄, MnSO₄, CaHPO₄, KCl, dried yeast(yeast containing Cr, yeast containing Se, yeast containing Mo), precipitated calcium carbonate, pomegranate extract, collagen, chitosan, green tea extract, Ginseng Radix extract, Acanthopanacis Cortex extract, onion extract, malic acid, citric acid, tartaric acid, fumaric acid, maleic acid and acetic acid.
